(19) United States
(12) Patent Application Publication
(10) Pub. No.: US 2011/0204222 A1
(43) Pub. Date: Aug. 25, 2011

(54) METHOD OF CHARACTERIZING PHYTOCHEMICALS FROM TRIGONELLA FOENUM GRACEUM

(75) Inventors: Villoo Morawala Patell, Karnataka (IN); Renuka Jain, Karnataka (IN); Manohar Shinde, Karnataka (IN); Henjarappa Jagadeesh Badamaranahalli, Karnataka (IN)

(73) Assignee: AVESTHAGEN LIMITED, Bangalore, Karnataka (IN)

(21) Appl. No.: 13/126,642

(22) PCT Filed: Oct. 28, 2009
(86) PCT No.: PCT/IB09/07261

§ 371 Date: Apr. 28, 2011

(30) Foreign Application Priority Data
Oct. 28, 2008 (IN) 2618/CHE/2008

(51) Int. Cl. H01J 49/26

(52) U.S. Cl. 250/282

(57) ABSTRACT

The present invention relates to identification and characterization of Phytochemicals and metabolites from Trigonella foenum-graceum extract by Liquid chromatography and Mass spectrometry LC-MS/MS.

### FIELD OF THE INVENTION

The present invention relates to identification and characterization of Phytochemicals and metabolites from Trigonella foenum-graceum extract by Liquid chromatography and Mass spectrometry LC-MS/MS.

BACKGROUND AND PRIOR ART OF THE INVENTION

Teestar™ is an extract of Fenugreek seeds. The plant is grown as green leafy vegetable and for its seeds. The plant is eaten as salad and also after cooking. The seed is a popular spice. The herb has light green leaves and produces slender beaked pods, which, consists of 10 to 20 3 mm long yellow hard seeds. India is one of the major producer and exporter of fenugreek. The seeds of fenugreek is used as medicine and consumed in various forms such as, Fenugreek tea. Fenugreek seeds are used to lower blood sugar levels, cholesterol management, remove dandruff, skin soothing, and to increase the milk produce in nursing mothers. Fenugreek contains good amount of protein, fat, fiber, carbohydrates, total ash, calcium, phosphorus, iron, sodium, potassium, vitamin B1, vitamin B2, niacin, vitamin C, vitamin A, and is particularly rich in fiber, gums and mucilage. The seed also contains various other phyto-chemicals such as Trigonellin, fenugreekin, hydroxyproline, flavonoids etc. The Fenugreek seeds contain an important constituent as gum-polysaccharide, polygalactomannan (PGM). It is a polymer of straight chains of mannose residues; every mannose residue is linked to its adjacent mannose by β1-4 glycosidic bonds, every mannose residue of the main chain is branched by α-D-galactose by α, 1-6 glycosidic bond. The ratio of mannose to galactose in Fenugreek seeds is 1:1 where as it is 1.6 in guar and 3.4 in locust bean.

In the present investigation metabolomics liquid chromatography (LC-MS/MS) approach has been used to identify and characterize the metabolites present in this plant. Metabolomics, a new “omics,” joining genomics, transcriptomics, and proteomics as a tool employed toward the understanding of global systems biology, has become widespread since 2002. Metabolomics focuses on the comprehensive and quantitative study of metabolites in a biological system. In contrast to genomics, transcriptomics and proteomics which, address macromolecules with similar chemical properties, such as DNA, RNA and proteins, metabolomics analysis deals with diverse properties of low molecular weight bio-compounds. Metabolomics offers a means of deciphering cellular metabolism and metabolic regulation. As metabolomics is the downstream product of genomics and proteomics, metabolomics is also complement of other “omics” for interpretation of gene function (functional genomics). Due to a wide range of metabolites in the metabolic network, e.g., approximately 600 metabolites in Saccharomyces cerevisiae, 1692 metabolites in Bacillus subtilis and up to 200000 metabolites in plant kingdom, it is a very challenging task to establish analytical tools for identifying and quantifying all of them.

A typical metabolomics study includes the collection of samples of interest, which follows the extraction of small molecules (low molecular weight metabolites) from the sample and is analyzed using techniques that separate and quantitate the molecules of interest. The analysis of the spectrum of metabolites are carried out by sophisticated separation and analytical techniques however, more precisely the hypenation techniques such as HPLC-MS/MS (high resolution mass spectrometry), GC-MS/MS, HPLC-NMR, are frequently being used by numerous investigators. The greatest advantage of LC-MS for application to metabolomic studies in pharmacology and toxicology is its flexibility. Different combinations of mobile phase and columns make it possible to tailor separations to the compounds of interest, including chiral compounds when appropriate conditions are used. As a result, most compounds can be analyzed by LC-MS. Instruments exist that enable low, medium, or high mass accuracy, and linear ion traps can enable MSⁿ, providing fragmentation profiles specific for given molecules.

### OBJECTIVE OF THE INVENTION

The main objective of the present invention is to obtain a method for characterizing phytochemicals present in an extract obtained from Trigonella foenum-graecum

Another main objective of the present invention is the identification and characterization of various phytochemicals present in the Fenugreek seed, Trigonella foenum-graceum extract (Teestar™) by LC-MS/MS (Applied Biosystems, MDS SCIEX 4000 Q-Trap MS/MS synchronized with Shimadzu HPLC, Prominence). Teestar™ is the phyto-extract claimed for the management of Diabetes mellitus in humans.

The +EMS of Total ion chromatogram (TIC) by Electrpspray ionisation liquid chromatography mass spectrometry ESI LC-MS/MS spectrum showed the presence of 1028 ions and the −EMS of TIC showed 2210 iond in Teestar™ extract. More prominent were 183 metabolites in the water extract, 117 metabolites in methanol water (9:1) and 145 metabolites in Methanol, chloroform, water (6:2:2) extract. (Table 1, FIGS. 1-3) The 41 different metabolites were identified by MS/MS analysis. (Table 2) and Mass spectra of few important meatbolites are given in FIG. 6-15.

In Teestar™ an important constituent as gum-polysaccharide, polygalactomannan (PGM) is also characterized by Liquid Chromatography and Mass spectrometry analysis (LC-MS/MS). This polygalactomannan molecule has the molecular mass of to be 217 kDa (FIG. 4a,b) Galactomannan (FIG. 4c) is a polymer (n=1269) of straight chains of mannose residues; every mannose residue is linked to its adjacent mannose by β1-4 glycosidic bonds, every mannose residue of the main chain is branched by a D galactose by a, 1-6 glycosidic bond. The ratio of mannose to galactose in Fenugreek seeds is 1:1. LC-MS analysis of the hydrolyzed product (FIG. 5a,b,c,d) was mostly hexose monomer (FIG. 5d).

### BRIEF DESCRIPTION OF ACCOMPANYING FIGURES

FIG. 1: Total ion chromatogram (TIC) of Teestar™ water extract in (a) positive ionization mode (b) negative ionization mode

FIG. 2: Total ion chromatogram (TIC) Teestar™ Methanol: water extract in (a) positive ionization mode (b) negative ionization mode

FIG. 3: Total ion chromatogram (TIC) of Teestar™ Methanol: Chloroform: water extract in (a) positive ionization mode (b) negative ionization mode

FIG. 4 (a) Teestar™—Convoluted mass spectrum of polygalactomannan with multiple charges (b) Teestar™—Deconvoluted mass spectrum of polygalactomannan displaying molecular mass of 217 kDa (c) Galactomannan structure (n=1269)

FIG. 5: (a) Total ion current chromatogram of hydrolyzed Teestar™ Galactomanan, (b) Enhanced mass spectrum of hydrolyzed Teestar™ Galactomanan (c) Retention time of extracted Glactomanan ion (XIC of enhanced mass spectrum) (d) Enhanced Mass spectrum of D—mannose/galactose

FIG. 6: (a) Enhanced product ion mass spectrum of ascirbic acid acid of mass 176 (b) Enhanced product ion mass spectrum of dehydroascorbic acid of mass 174

FIG. 7: Enhanced product ion mass spectrum of Diosgenin of mass 413

FIG. 8: Enhanced product ion mass spectrum of Gentainin of mass 175.8

FIG. 9: (a) Enhanced product ion mass spectrum of Isovitexin of mass 431 (b) Enhanced product ion mass spectrum of Orientin of mass 447

FIG. 10: Enhanced product ion mass spectrum of Kaempferol of mass 285

FIG. 11: Enhanced product ion mass spectrum of Muurolene of mass 204

FIG. 12: Enhanced product ion mass spectrum of Tigogenin of mass 415

FIG. 13: Enhanced product ion mass spectrum of Trigonellin of mass 137

FIG. 14: (a) Enhanced product ion mass spectrum of 4-hydroxyiso leucine of mass 147 (b) Enhanced product ion mass spectrum of tryptophan of mass 204 (c) Enhanced product ion mass spectrum of 2,3-dihydroxybenzofurane of mass 120

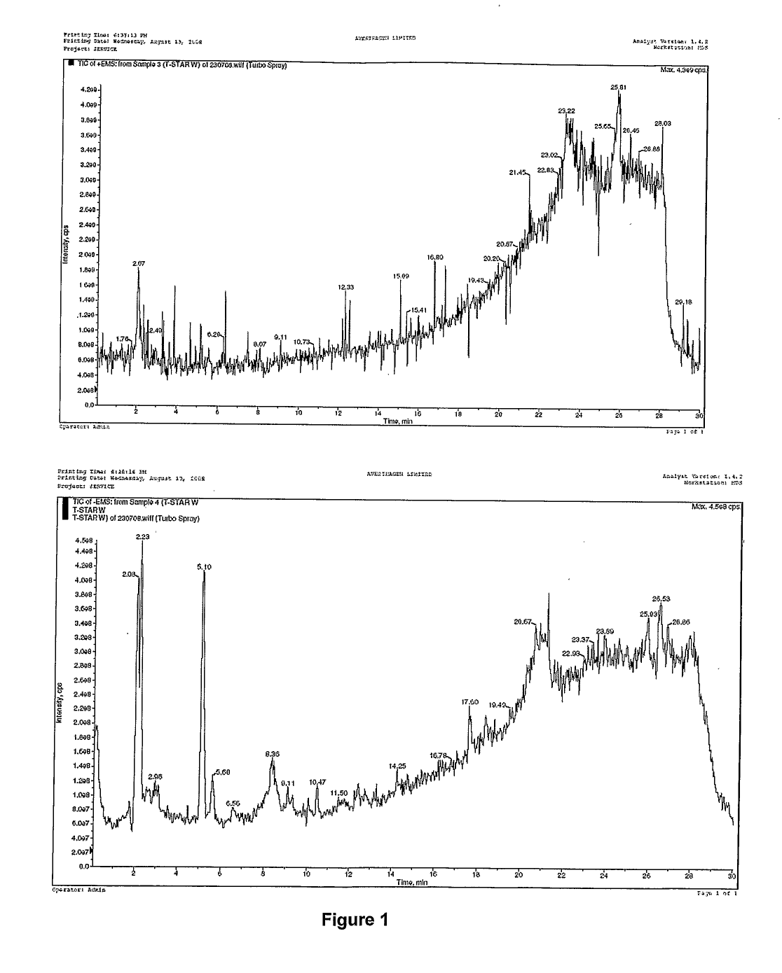

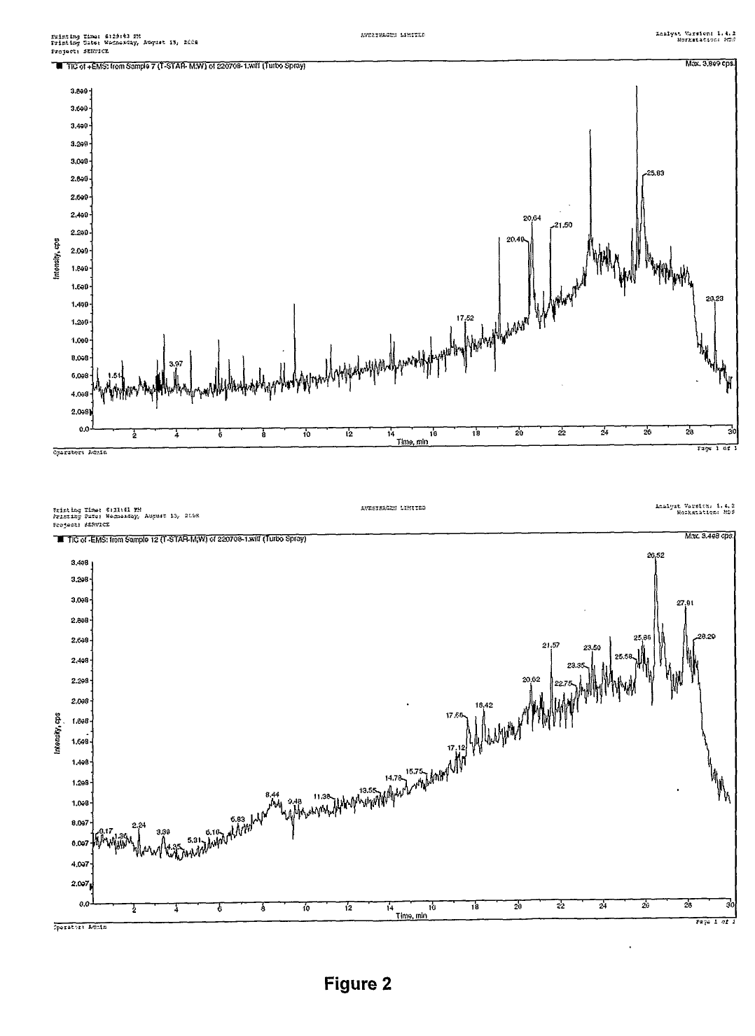

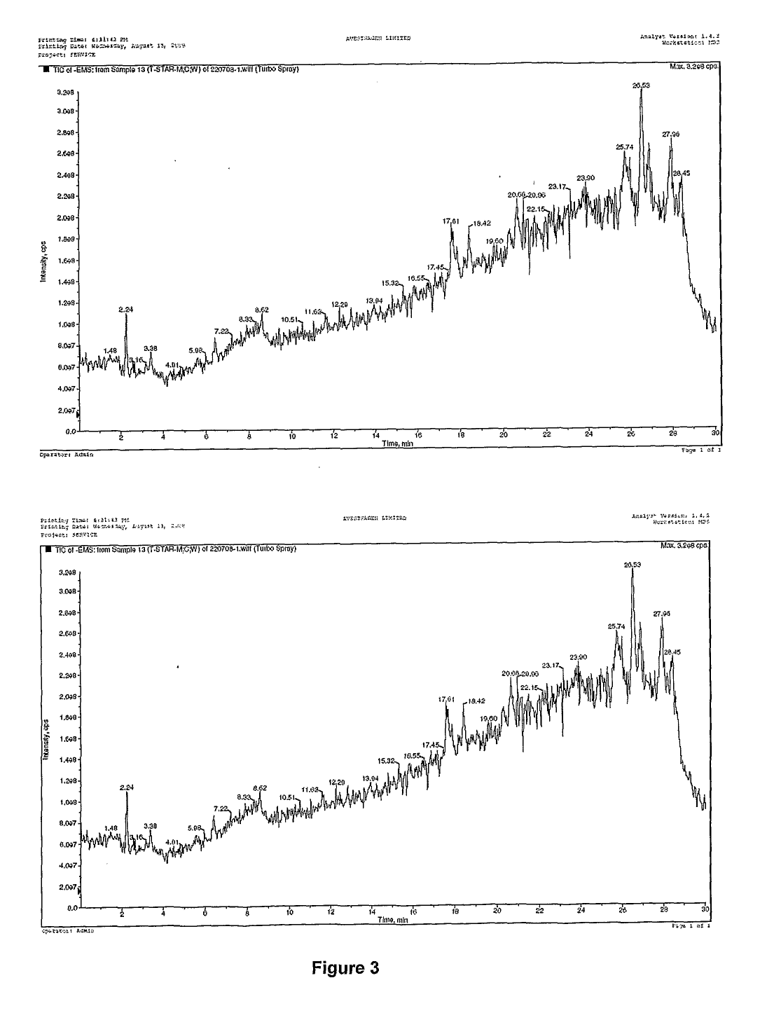

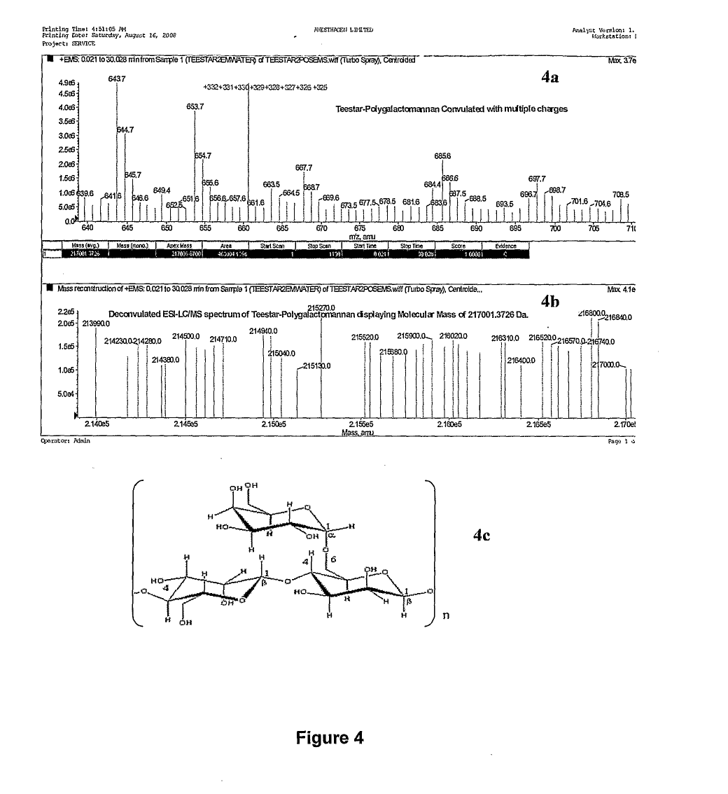

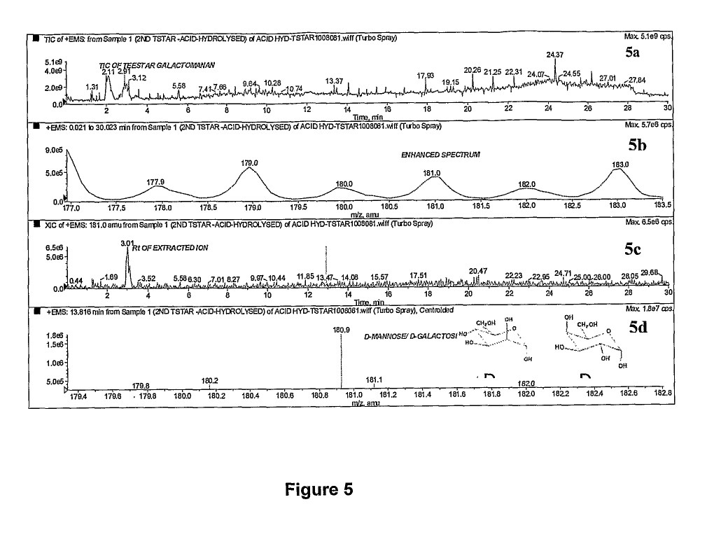

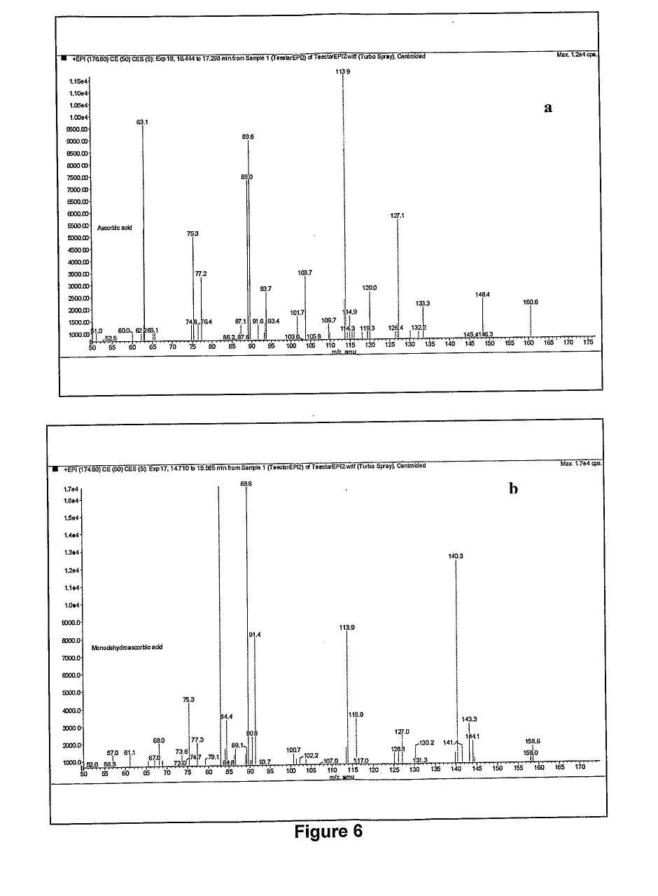

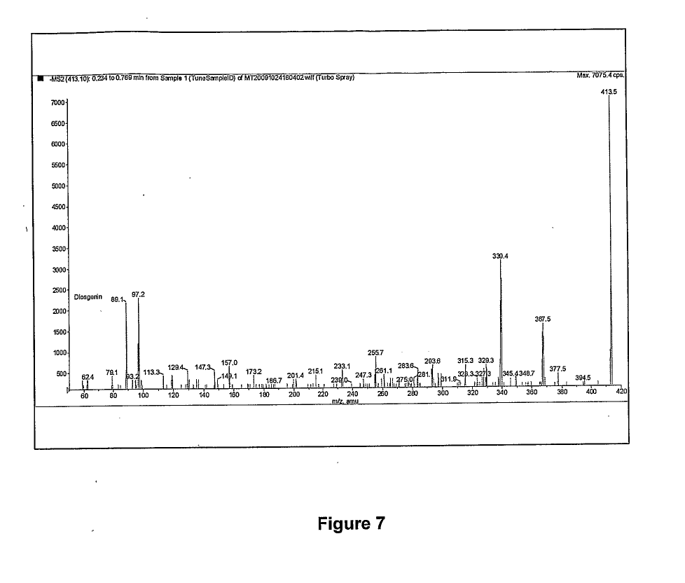

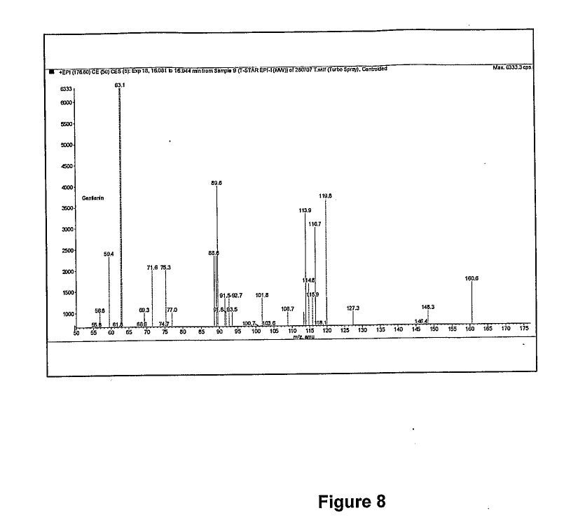

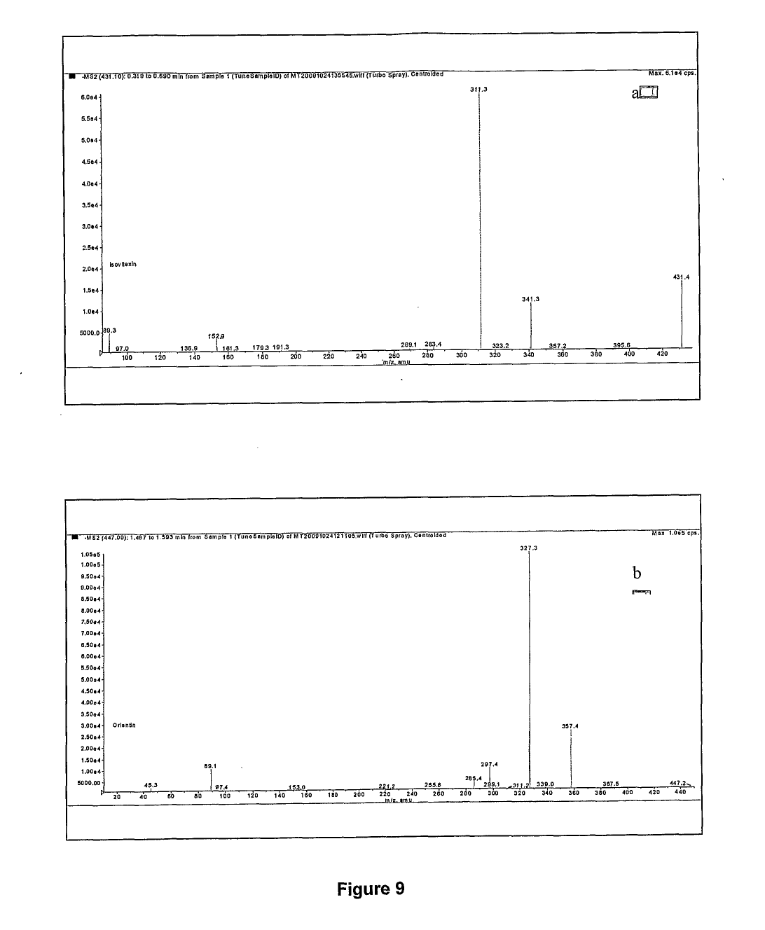

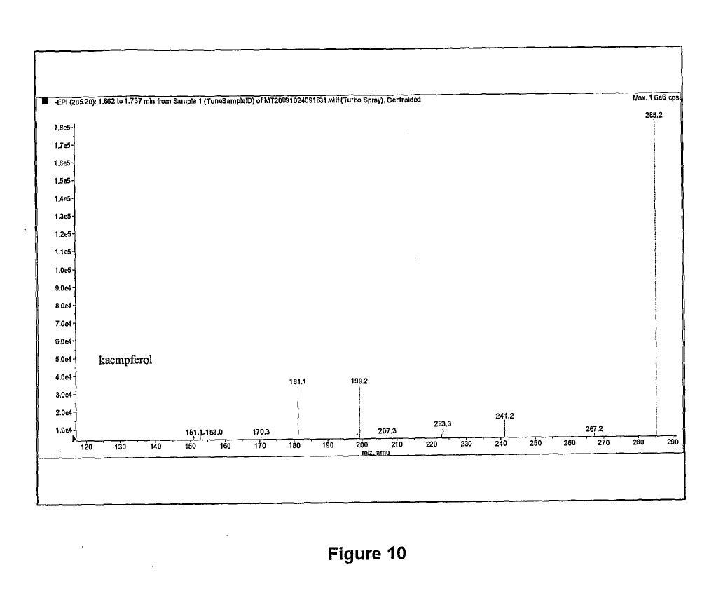

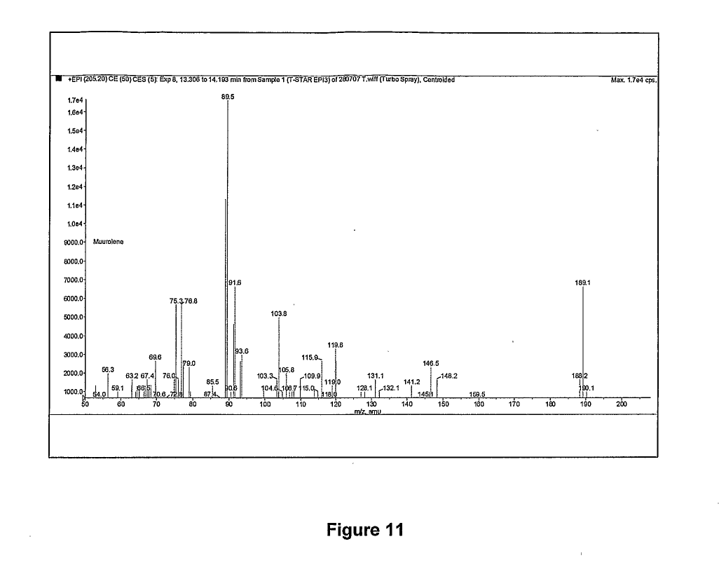

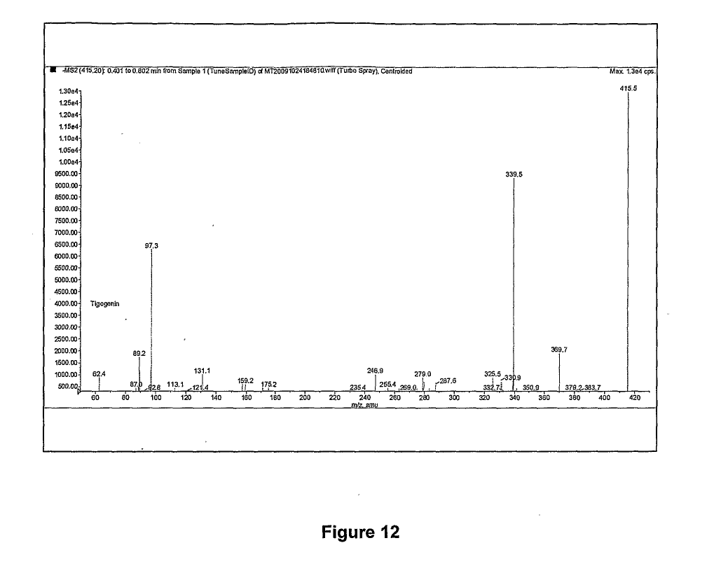

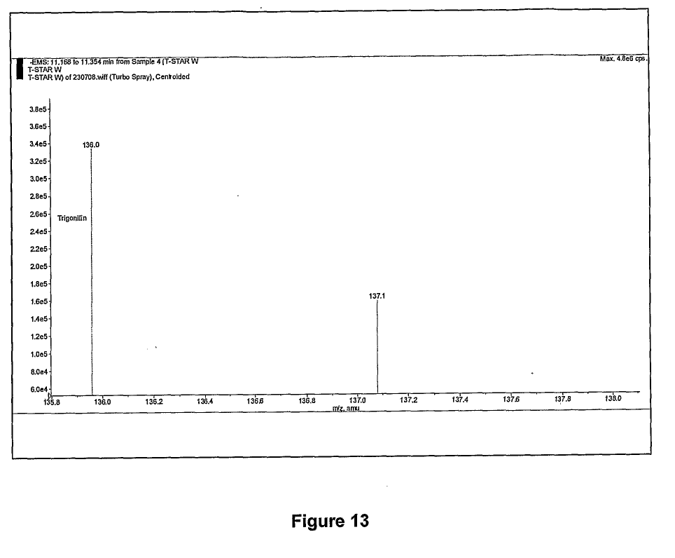

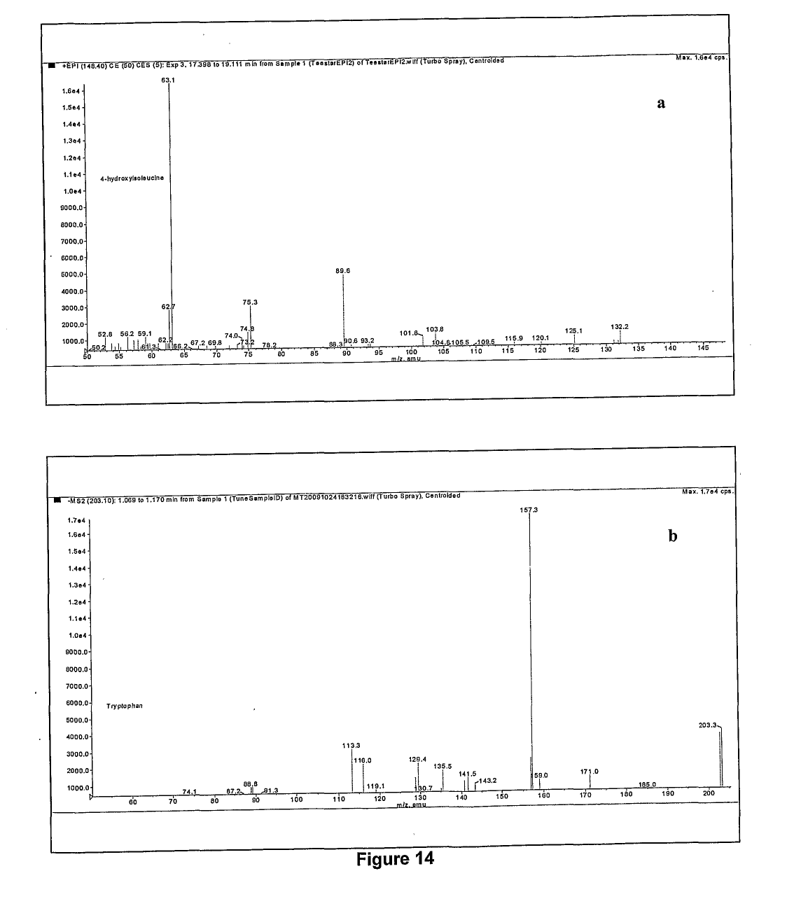

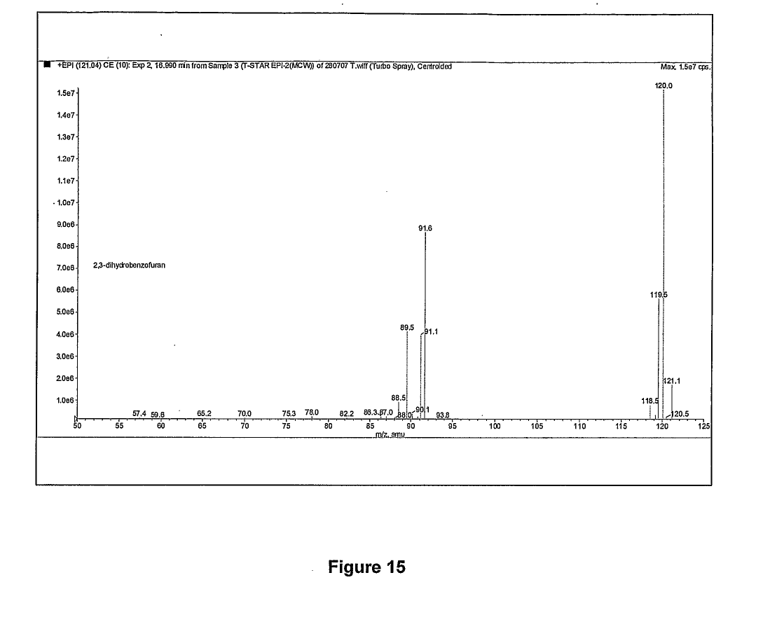

### DETAILED DESCRIPTION OF THE INVENTION

The present invention relates to a method for characterizing phytochemicals present in an extract, said method comprising steps of:
a) Preparing the sample for extraction of phytochemicals; and
b) subjecting the prepared sample to Liquid chromatography followed by Mass spectrometry.

In another embodiment of the present invention, the extract is a plant extract.

In yet another embodiment of the present invention, the extract is obtained from Trigonella species, preferably Trigonella foenum-graecum

In still another embodiment of the present invention the Mass Spectrometry is operated in positive polarity mode or negative polarity mode or a combination of positive and negative polarity modes.

In still another embodiment of the present invention the Liquid Chromatography is preferably High Performance Liquid Chromatography.

In still another embodiment of the present invention the phytochemicals are extracted using mixture of water, methanol or chloroform and combinations thereof.

In still another embodiment of the present invention the ratio for the mixture of methanol and water is preferably 9:1 respectively.

In still another embodiment of the present invention the ratio for the mixture of methanol, chloroform and water is preferably 6:2:2 respectively.

Analysis for the identification of various phytochemicals/metabolites present in Teestar™ by LC-MS/MS (Applied Biosystems MDS SCIEX 4000 Q Trap MS/MS)
i) Acquisition of enhanced mass spectrum in positive ionisation mode (+EMS) in full scan mode from m/z 50 amu to 1000 amu
ii) Acquisition of enhanced mass spectrum in negative ionisation mode (−EMS) in full scan mode from m/z 50 amu to 1000 amu
iii) Acquisition of MS/MS of selected ions

The standard operation procedure (SOP) describes
i) The preparation of Teestar™ sample
ii) Acquisition procedure by LC-MS/MS for the separation and detection of phytochemicals

The Teestar™ is an extract of fenugreek seeds. The plant is grown as green leafy vegetable and for its seeds. The plant is eaten as salad and also after cooking. The seed is a popular spice. The herb has light green leaves and produces slender beaked pods, which, consists of 10 to 20 3 mm long yellow hard seeds. India is one of the major producer and exporter of fenugreek. The seeds of fenugreek is used as medicine and consumed in various forms such as, Fenugreek tea. Fenugreek seeds are used to lower blood sugar levels, cholesterol management, remove dandruff, skin soothing, and to increase the milk produce in nursing mothers. Fenugreek contains good amount of protein, fat, fiber, carbohydrates, total ash, calcium, phosphorus, iron, sodium, potassium, vitamin B1, vitamin B2, niacin, vitamin C, vitamin A, and is particularly rich in fiber, gums and mucilage. The seed also contains various other phyto-chemicals such as Trigonellin, fenugreekin, hydroxyproline, flavonoids etc. The Fenugreek seeds contain an important constituent as gum-polysaccharide, polygalactomannan (PGM). It is a polymer of straight chains of mannose residues; every mannose residue is linked to its adjacent mannose by β1-4 glycosidic bonds, every mannose residue of the main chain is branched by α-D-galactose by α, 1-6 glycosidic bond. The ratio of mannose to galactose in Fenugreek seeds is 1:1 where as it is 1.6 in guar and 3.4 in locust bean.

### Sample Preparation:

### Extraction of Phytochemicals:

4 mg of Teestar™ sample(s) were weighed in three clean sterilized 1.5 ml graduated vials and 1 mL of water was added to vial 1,1 mL of methanol: water (9:1) to vial 2, 1 mL of methanol, chloroform, water (6:2:2) to vial 3 respectively. The sample in vial was, incubated for 16 hours at 8° C. At the end of the incubation time the sample was placed in a hot water bath for 10 min The contents of the vials 2 and 3 were mixed thoroughly by a vortex for 5 min. further; the vials were placed in a sonicator bath for 1 hour and were centrifuged for 15 min at 14000 rpm and 4° C. to remove any suspended particles. 500 μL of the centrifuged extract was filtered through a 0.22μ syringe filter. The filtered extract were carefully transferred into 1.5 mL autosampler vials (Shimadzu Prominence). HPLC autosampler (Shimadzu, SIL20AC).

### Solubilization of Polygalactomannan for the Determination of Molecular Mass by ESI-LC/MS/MS:

100 mg of Teestar™ sample was suspended into a 50 ml conical flask, washed with methanol, followed by petroleum ether, followed by chloroform. The extract was dried in vacuum and was further washed in hot methanol. The sample was filtered and dried in vacuum. The sample was then placed in a conical flask containing 10 ml of water (ultra pure, Milli-Q water). The mixture was allowed to dissolve/swell for 4 hours. At the end of the incubation time the flask containing swollen Teestar powder was transferred to a boiling water bath for exactly 10 min. A 1-ml of the processed sample was transferred to a 1.5 ml graduated Ependorof vial.

This was centrifuged for 15 min at 14000 rpm and 4° C. The sample was then filtered through a 0.2μ syringe filter and the clear filtrate was carefully transferred to an auto sampler vial.

### Digestion of Polygalactomannan for the Determination of its Monomeric Molecular Mass by ESI-LC/MS/MS:

100 mg of Teestar™ sample was suspended into a 50 mL conical flask, the sample was processed as shown above. The processed sample was then added into a conical flask containing 10 ml of dilute HCL (pH 2, HCL in ultra pure, Milli-Q water). The mixture was allowed to dissolve/swell for 2 hours at 50° C. in a temperature controlled water bath while brief stirring (2 minutes) at every 15 minutes interval. The mixture was then transferred to a boiling water bath for exactly 3 hours. The viscous solution formed was allowed to cool and was centrifuged for 30 min. at 14000 rpm and 20° C. The acid hydrolyzed Teestar™ solution was filtered through a 0.22μ filter and 1 mL of the processed filtrate transferred to an autosampler vial.

### LC-MS/MS Analysis:

All the extracts sample were filtered through a 0.2-μ-syringe filter, the clarified extracts were carefully transferred into respective autosampler vials (1.5 mL capacity, autosampler (SIL20AC) attached to HPLC (Shimadzu, Prominence). The blank of water, methanol:water (9:1) and methanol:chloroform:water (6:2:2) were added into respective vials. The temperature of the autosampler was maintained at 8° C. throughout the experiment. The samples were eluted from HPLC by a binary gradient through a 5μ particle size RP-18 column, (4.6 mm D×250 mm×L) held at 40° C. in a temperature controlled column oven (CTO 20AC) at a flow rate of 1 ml/min over 30.01 min. The gradient system consisted of 0.1% aqueous formic acid (A) and 0.1% formic acid in acetonitrile (B). The gradient was programmed to attain 75% (B) over 20 min, remains same till 25 min and decreases instantly to 5% at the end of 26 min. The 5% (B) remains till 30 min and the HPLC stops at 31.01 min. The HPLC eluent was subjected into mass spectrometer (Applied Biosystems MDS SCIEX 4000 Q Trap MS/MS) by a splitter. The Mass spectrometer was operated by attaching a splitter in an EMS positive and negative polarity mode with ion spray voltage 2750, source temperature 350° C., vacuum 4.6⁻⁵ Torr, curtain gas 20, Collision Energy (CE) 10.00, Collision Energy spread (CES) 10.000, GS1 40, GS2 60, collision energy 10 and declusteuring potential of 35. The turbo ion source was set at 1000 amu/s with the interface heater ‘on’, 967 scans in a period and LIT fill time 20 m sec and dynamic LIT fill time on.

### Acquisition of Enhanced Product Ion EPI by LC-MS/MS—

The enhanced product ion and MS/MS was performed at LC flow rate of 1 mL min⁻¹ over a period of 30.01 min, in splitter-attached mode. The MS was operated both in positive and negative polarity mode. For positive polarity mode the curtain gas was set to 20, Collision Energy 40, CES 10, ion spray voltage was set at 4000.00 GS1 40, GS2 60 with interface heater and the dynamic fill time on. For negative polarity mode the curtain gas was set to 20, Collision Energy -40, CES 10, ion spray voltage was set at −4000.00, temp 400.00, GS1 40, GS2 60 with interface heater and the dynamic fill time on.

For the processing, the total ion chromatogram (TIC) of blank (solvent) and test sample were Gaussian smooth, base line subtracted and noise was set to 1%. The TIC of blank was subtracted from that of the TIC of test and the spectrum was generated using Analyst Software 1.4.2. The noise level of spectrum was set to 1%. The processed spectrum is also manually verified. The data list is then generated to check the number of ions present with their m/z, centroid m/z, peak intensities, resolution, peak area and their charge specification. Next level of processing involves the elimination of the multiple charge ions by checking their singly charged ions. The low intense ions are further extracted to obtain Extracted ion chromatogram (XIC) or amplified.

TABLE 1
Mass peak list of Teestar extracted by various solvent
Mass peak list of Teestar extracted by various solvent

| | | Mass peak | | Mass peak list | |
|---|---|---|---|---|---|
| | | list Teestar | | Teestar extracted | |
| | | extracted with | | with Methanol: | |
| Mass peak list Teestar | | Methanol:water | | Chloroform:water | |
| extracted with water | | (9:1) | | (6:2:2) | |
| | Centroid | | Centroid | | Centroid |
| | mass | | mass | | mass |
| m/z (amu) | (amu) | m/z (amu) | (amu) | m/z (amu) | (amu) |
| 52.1653 | 52.1653 | 86.48 | 86.4832 | 75.6 | 75.6386 |
| 55.0283 | 55.0283 | 87.52 | 87.4945 | 76.24 | 76.2551 |
| 59.0489 | 59.0489 | 92.16 | 92.1341 | 76.88 | 76.8927 |
| 62.1033 | 62.1033 | 92.56 | 92.5975 | 77.36 | 77.3361 |
| 62.8 | 62.8 | 95.6 | 95.5773 | 77.92 | 77.9222 |
| 63.6798 | 63.6798 | 98.72 | 98.7001 | 78.4 | 78.3727 |
| 66.2411 | 66.2411 | 100.24 | 100.249 | 79.36 | 79.3753 |
| 69.6285 | 69.6285 | 100.8 | 100.7795 | 86.56 | 86.5356 |
| 71.2629 | 71.2629 | 101.76 | 101.7701 | 87.6 | 87.4195 |
| 78.3791 | 78.3791 | 106.8 | 106.8114 | 92.16 | 92.1241 |
| 79.9085 | 79.9085 | 107.84 | 107.831 | 92.64 | 92.5976 |
| 82.4719 | 82.4719 | 111.92 | 111.8665 | 98.72 | 98.7056 |
| 83.4098 | 83.4098 | 112.88 | 112.8973 | 99.44 | 99.4776 |
| 87.9987 | 87.9987 | 116 | 115.9801 | 100.24 | 100.2565 |
| 88.5092 | 88.5092 | 120.48 | 120.4503 | 100.8 | 100.7785 |
| 92.5942 | 92.5942 | 121.04 | 121.0368 | 101.84 | 101.7865 |
| 94.1545 | 94.1545 | 122 | 122.0397 | 106.8 | 106.822 |
| 95.6 | 95.6 | 130.08 | 130.15 | 107.84 | 107.8538 |
| 96.5854 | 96.5854 | 135.28 | 135.2566 | 112.88 | 112.906 |
| 97.6486 | 97.6486 | 136.32 | 136.2861 | 116 | 115.9627 |
| 98.1647 | 98.1647 | 144.32 | 144.3473 | 120.48 | 120.4526 |
| 98.6827 | 98.6827 | 147.68 | 147.6594 | 121.04 | 121.0371 |
| 99.1988 | 99.1988 | 148.4 | 148.4015 | 122.08 | 122.0327 |
| 99.6963 | 99.6963 | 149.44 | 149.4329 | 127.2 | 127.1673 |
| 100.2609 | 100.261 | 150.48 | 150.4908 | 130.16 | 130.1683 |
| 100.78 | 100.78 | 152.56 | 152.5374 | 134.16 | 134.2373 |
| 102.6977 | 102.698 | 156.48 | 156.5505 | 135.28 | 135.2556 |
| 103.2407 | 103.241 | 157.6 | 157.6063 | 144.4 | 144.3971 |
| 103.44 | 103.44 | 158.48 | 158.5392 | 146.48 | 146.4025 |
| 104.2386 | 104.239 | 160.64 | 160.6337 | 147.68 | 147.7068 |
| 105.268 | 105.268 | 162.64 | 162.6586 | 148.4 | 148.4137 |
| 106.3004 | 106.3 | 164.72 | 164.7133 | 149.44 | 149.4458 |
| 107.2993 | 107.299 | 166.72 | 166.6819 | 150.48 | 150.4961 |
| 108.8015 | 108.802 | 170.8 | 170.791 | 152.56 | 152.5331 |
| 110.865 | 110.865 | 172.8 | 172.8014 | 153.6 | 153.5672 |
| 111.3422 | 111.342 | 173.84 | 173.8254 | 154.56 | 154.5259 |
| 111.8475 | 111.848 | 174.8 | 174.8385 | 155.6 | 155.5407 |
| 114.8565 | 114.857 | 176.8 | 176.8391 | 156.56 | 156.5651 |
| 115.9939 | 115.994 | 178.88 | 178.8871 | 157.6 | 157.6109 |
| 116.9548 | 116.955 | 189.04 | 189.0571 | 158.56 | 158.5473 |
| 118.9652 | 118.965 | 191.04 | 191.0716 | 160.64 | 160.6536 |
| 120.4556 | 120.456 | 195.12 | 195.1291 | 162.64 | 162.6576 |
| 121.0338 | 121.034 | 199.2 | 199.1815 | 164.72 | 164.7097 |
| 124.9937 | 124.994 | 201.2 | 201.1989 | 166.72 | 166.6957 |
| 126.5499 | 126.55 | 203.2 | 203.2032 | 170.8 | 170.8139 |
| 127.1337 | 127.134 | 204.24 | 204.2213 | 171.6 | 171.7459 |
| 129.2579 | 129.258 | 204.88 | 204.8494 | 172.8 | 172.8202 |
| 130.1077 | 130.108 | 205.2 | 205.1807 | 173.84 | 173.83 |
| 131.1203 | 131.12 | 206.24 | 206.2031 | 174.88 | 174.862 |
| 131.5869 | 131.587 | 207.28 | 207.2635 | 176.88 | 176.8416 |
| 133.2641 | 133.264 | 208.32 | 208.2814 | 178.88 | 178.8983 |
| 143.3584 | 143.358 | 209.28 | 209.2549 | 186.08 | 186.0736 |
| 145.3921 | 145.392 | 211.28 | 211.2527 | 186.96 | 187.0135 |
| 151.5224 | 151.522 | 212.24 | 212.2335 | 189.04 | 189.0707 |
| 159.5971 | 159.597 | 213.28 | 213.2498 | 191.12 | 191.0801 |
| 161.71 | 161.71 | 214.32 | 214.2843 | 192.08 | 192.1097 |
| 163.7042 | 163.704 | 215.28 | 215.2801 | 193.12 | 193.1152 |
| 165.7613 | 165.761 | 217.28 | 217.2633 | 195.12 | 195.1478 |
| 168.7853 | 168.785 | 219.36 | 219.3267 | 199.2 | 199.1848 |
| 188.0536 | 188.054 | 221.36 | 221.3257 | 200.24 | 200.2272 |
| 190.0441 | 190.044 | 223.28 | 223.2954 | 201.2 | 201.2005 |
| 204.3047 | 204.305 | 224.32 | 224.2925 | 203.2 | 203.2195 |
| 218.3083 | 218.308 | 225.36 | 225.3504 | 204.24 | 204.238 |
| 276.6549 | 276.655 | 227.36 | 227.3851 | 204.88 | 204.88 |
| 327.9133 | 327.913 | 228.48 | 228.419 | 205.2 | 205.1902 |
| 341.9293 | 341.929 | 229.04 | 229.047 | 206.24 | 206.2342 |
| 342.9205 | 342.921 | 229.36 | 229.3802 | 207.28 | 207.2747 |
| 423.7689 | 423.769 | 230.4 | 230.3874 | 208.24 | 208.2598 |
| 494.7218 | 494.722 | 231.36 | 231.3853 | 209.28 | 209.2487 |
| 537.2256 | 537.226 | 233.36 | 233.3753 | 211.28 | 211.2549 |
| 550.3416 | 550.342 | 235.44 | 235.4433 | 212.24 | 212.2403 |
| 565.4409 | 565.441 | 237.44 | 237.4554 | 213.28 | 213.2593 |
| 566.3944 | 566.394 | 239.44 | 239.4385 | 214.32 | 214.3163 |
| 610.2104 | 610.21 | 245.52 | 245.4816 | 215.28 | 215.2753 |
| 613.284 | 613.284 | 247.6 | 247.5613 | 217.28 | 217.2748 |
| 626.6305 | 626.631 | 249.52 | 249.5356 | 219.36 | 219.3556 |
| 637.3591 | 637.359 | 251.52 | 251.5196 | 220.4 | 220.3643 |
| 638.3589 | 638.359 | 252.56 | 252.5768 | 221.36 | 221.326 |
| 649.3461 | 649.346 | 253.52 | 253.5325 | 223.28 | 223.3164 |
| 659.301 | 659.301 | 255.52 | 255.5598 | 224.32 | 224.3041 |
| 660.3543 | 660.354 | 257.52 | 257.5666 | 225.36 | 225.3358 |
| 675.2987 | 675.299 | 261.52 | 261.5473 | 227.36 | 227.3759 |
| 676.3316 | 676.332 | 263.6 | 263.5911 | 228.48 | 228.475 |
| 682.448 | 682.448 | 265.6 | 265.6341 | 229.36 | 229.3868 |
| 684.3217 | 684.322 | 267.6 | 267.6412 | 230.4 | 230.3975 |
| 719.5109 | 719.511 | 269.68 | 269.6511 | 231.36 | 231.3883 |
| 758.2151 | 758.215 | 271.6 | 271.6508 | 233.36 | 233.3866 |
| 759.3251 | 759.325 | 273.68 | 273.6479 | 235.44 | 235.4342 |
| 780.4293 | 780.429 | 277.68 | 277.6761 | 237.44 | 237.4621 |
| 791.4022 | 791.402 | 278.72 | 278.72 | 239.44 | 239.4485 |
| 794.4892 | 794.489 | 279.68 | 279.651 | 240.48 | 240.4478 |
| 806.4855 | 806.486 | 280.72 | 280.6824 | 241.44 | 241.4773 |
| 807.4224 | 807.422 | 281.68 | 281.7013 | 242.56 | 242.5486 |
| 809.4563 | 809.456 | 282.8 | 282.8079 | 245.44 | 245.4688 |
| 811.324 | 811.324 | 283.84 | 283.8067 | 247.52 | 247.5295 |
| 812.362 | 812.362 | 284.8 | 284.8304 | 249.52 | 249.5315 |
| 818.4137 | 818.414 | 285.76 | 285.7476 | 251.52 | 251.5266 |
| 821.4355 | 821.436 | 291.76 | 291.7893 | 252.56 | 252.5802 |
| 822.458 | 822.458 | 293.84 | 293.7859 | 253.52 | 253.5468 |
| 823.4573 | 823.457 | 299.84 | 299.8459 | 255.6 | 255.5918 |
| 824.4535 | 824.454 | 301.76 | 301.7344 | 256.64 | 256.6377 |
| 826.4353 | 826.435 | 304.96 | 304.9462 | 257.6 | 257.5738 |
| 827.4392 | 827.439 | 327.84 | 327.8185 | 259.52 | 259.5596 |
| 828.3525 | 828.353 | 338.96 | 338.9571 | 261.6 | 261.5603 |
| 830.4325 | 830.433 | 382.48 | 382.4601 | 265.68 | 265.6589 |
| 832.3438 | 832.344 | 383.44 | 383.4447 | 267.68 | 267.6503 |
| 833.3163 | 833.316 | 415.2 | 415.1975 | 269.68 | 269.6939 |
| 834.341 | 834.341 | 416.16 | 416.1898 | 271.68 | 271.6797 |
| 835.3515 | 835.352 | 437.12 | 437.1449 | 273.68 | 273.6595 |
| 836.4263 | 836.426 | 438.16 | 438.1343 | 277.68 | 277.7 |
| 837.4505 | 837.451 | 453.12 | 453.0954 | 278.72 | 278.7373 |
| 838.4259 | 838.426 | 460.24 | 460.2065 | 279.68 | 279.6631 |
| 842.4318 | 842.432 | 536.16 | 536.1132 | 280.64 | 280.665 |
| 843.4285 | 843.429 | 610.16 | 610.1448 | 281.68 | 281.7145 |
| 844.4115 | 844.412 | 637.28 | 637.2772 | 282.8 | 282.8202 |
| 846.4354 | 846.435 | 638.24 | 638.2815 | 283.76 | 283.7816 |
| 848.462 | 848.462 | | | 284.8 | 284.81 |
| 849.4254 | 849.425 | | | 285.76 | 285.7523 |
| 850.3712 | 850.371 | | | 291.84 | 291.8155 |
| 856.4404 | 856.44 | | | 293.84 | 293.8047 |
| 862.4088 | 862.409 | | | 299.84 | 299.8265 |
| 863.467 | 863.467 | | | 301.84 | 301.8015 |
| 866.431 | 866.431 | | | 304.96 | 304.9716 |
| 872.4366 | 872.437 | | | 306 | 305.968 |
| 873.4175 | 873.418 | | | 309.84 | 309.8947 |
| 874.4652 | 874.465 | | | 320 | 320.0075 |
| 875.4514 | 875.451 | | | 327.84 | 327.8366 |
| 876.4632 | 876.463 | | | 338.96 | 338.9398 |
| 878.4697 | 878.47 | | | 382.48 | 382.4502 |
| 880.4669 | 880.467 | | | 393.12 | 393.0869 |
| 883.4379 | 883.438 | | | 397.2 | 397.1934 |
| 883.92 | 883.92 | | | 415.2 | 415.162 |
| 884.4068 | 884.407 | | | 416.16 | 416.1726 |
| 885.373 | 885.373 | | | 437.12 | 437.129 |
| 886.3748 | 886.375 | | | 438.08 | 438.0806 |
| 888.4027 | 888.403 | | | 453.04 | 453.0677 |
| 889.3917 | 889.392 | | | 460.16 | 460.195 |
| 890.4128 | 890.413 | | | 536.16 | 536.1034 |
| 892.4411 | 892.441 | | | 546.4 | 546.3801 |
| 894.4136 | 894.414 | | | 590.4 | 590.4038 |
| 899.4643 | 899.464 | | | 610.16 | 610.1438 |
| 900.5056 | 900.506 | | | 611.12 | 611.1375 |
| 902.4787 | 902.479 | | | 612.16 | 612.1587 |
| 906.4797 | 906.48 | | | 637.28 | 637.2392 |
| 907.3911 | 907.391 | | | | |
| 908.3108 | 908.311 | | | | |
| 912.3521 | 912.352 | | | | |
| 913.4054 | 913.405 | | | | |
| 916.96 | 916.96 | | | | |
| 921.4268 | 921.427 | | | | |
| 922.4277 | 922.428 | | | | |
| 926.4268 | 926.427 | | | | |
| 929.84 | 929.84 | | | | |
| 930.3887 | 930.389 | | | | |
| 931.3625 | 931.363 | | | | |
| 932.4354 | 932.435 | | | | |
| 943.3544 | 943.354 | | | | |
| 944.3564 | 944.356 | | | | |
| 949.4486 | 949.449 | | | | |
| 950.4462 | 950.446 | | | | |
| 956.4446 | 956.445 | | | | |
| 958.0819 | 958.082 | | | | |
| 962.4624 | 962.462 | | | | |
| 972.3492 | 972.349 | | | | |
| 973.1861 | 973.186 | | | | |
| 974.1656 | 974.166 | | | | |
| 975.2855 | 975.286 | | | | |
| 977.5205 | 977.521 | | | | |
| 978.3327 | 978.333 | | | | |
| 978.8041 | 978.804 | | | | |
| 980.0837 | 980.084 | | | | |
| 981.12 | 981.12 | | | | |
| 981.4612 | 981.461 | | | | |
| 982.4135 | 982.414 | | | | |
| 983.3869 | 983.387 | | | | |
| 990.2488 | 990.249 | | | | |
| 990.8 | 990.8 | | | | |
| 991.2995 | 991.3 | | | | |
| 992.3209 | 992.321 | | | | |
| 994.4034 | 994.403 | | | | |
| 995.2236 | 995.224 | | | | |
| 996.2789 | 996.279 | | | | |

TABLE 2
Metabolites identified from Teestar ™

| Sl. No. | Name of the Compound | Mol. Wt. |
|---|---|---|
| 1 | Ascrobic acid | 175.1 |
| 2 | Glutamic acid | 146.0029 |
| 3 | Glycine | 75 |
| 4 | Histidine | 155 |
| 5 | Isoleucine | 131.59 |
| 6 | Leucine | 131 |
| 7 | Lysine | 145/146/147 |
| 8 | Niacian | 124/123 |
| 9 | Riboflavin | 342 |
| 10 | Serine | 106.3/105.3 |
| 11 | Thiamine | 246.05 |
| 12 | Tryptophan | 203.1/204.09 |
| 13 | Tyrosine | 180.04/181.01 |
| 14 | Valine | 116.07/117.0 |
| 15 | Cysteine | 122 |
| 16 | Aspartic acid | 134 |
| 17 | Arginine | 174.8 |
| 18 | Alanine | 89 |
| 19 | Gitoginin | 431.1861 |
| 20 | Isovitexin | 432 |
| 21 | Leuteoline | 286.07 |
| 22 | Muurolene | 204.3/205.2 |
| 23 | Quercetin | 302.4 |
| 24 | Trigonellin | 136.0064 |
| 25 | Pentose | 149.4 |
| 26 | Hexose | 180 |
| 27 | 4-Hydroxy isoleucine | 147 |
| 28 | Biotin | 245 |
| 29 | Disaccharide | 342 |
| 30 | Trisaccharide | 503/504 |
| 31 | Dihydrobenzofuran | 121 |
| 32 | Gentianin | 175 |
| 33 | Palmatic acid | 255 |
| 34 | Elemene | 287 |
| 35 | Diosgenin | 413 |
| 36 | Carpaine | 471.377 |
| 37 | Glycerol | 92.05 |
| 38 | 3hydroxy 4,5, dimethyl 2-furanone | 127.2 |
| 39 | Orientin | 447 |
| 40 | Tigogenin | 415 |
| 41 | Monodehydroascorbic acid | 174 |

We claim:

1. A method for characterizing phytochemicals present in an extract, said method comprising steps of:
a) sample preparation for extraction of phytochemicals; and metabolotes
b) Liquid chromatography and Mass spectrometry.

2. The method as claimed in claim 1, wherein the extract is a plant extract.

3. The method as claimed in claim 2, wherein the extract is obtained from Trigonella species, preferably Trigonella foenum-graecum

4. The method as claimed in claim 1, wherein the Mass Spectrometry is operated in positive polarity mode or negative polarity mode or a combination of positive and negative polarity modes.

5. The method as claimed in claim 1, where the Liquid Chromatography is preferably High Performance Liquid Chromatography.

6. The method as claimed in claim 1, wherein the phytochemicals are extracted using mixture of water, methanol or chloroform and combinations thereof.

7. The method according to claim 5, wherein the ratio for the mixture of methanol and water is preferably 9:1 respectively.

8. The method as claimed in claim 5, wherein the ratio for the mixture of methanol, chloroform and water is preferably 6:2:2 respectively.

9. The method as claimed in claim 1, for characterization of polygalactomannan

10. The method as claimed in claim 1 for characterization of phytochemicals from Trigonella foenum graceum extract.
